(19) United States
(12) Patent Application Publication
(10) Pub. No.: US 2015/0045858 A1
(43) Pub. Date: Feb. 12, 2015

(54) Brain Cooling Device for Craniotomy Patient

(71) Applicant: Kyungpook National University Industry-Academic Cooperation Foundation, Daegu (KR)

(72) Inventors: Jae Chan Park, Daegu (KR); Kyoung Ho Suk, Daegu (KR); Hyung Soo Han, Daegu (KR); Dong Ick Rhee, Gyeonggi-do (KR)

(73) Assignee: Kyungpook National University Industry-Academic Cooperation Foundation, Daegu (KR)

(21) Appl. No.: 14/375,276

(22) PCT Filed: Feb. 6, 2013
(86) PCT No.: PCT/KR13/00959

§ 371 Date: Jul. 29, 2014

(30) Foreign Application Priority Data
Feb. 15, 2012 (KR) 10-2012-0015558

(51) Int. Cl. A61F 7/10

(52) U.S. Cl. CPC A61F 7/10; A61F 2007/0002
USPC 607/105; 607/113

(57) ABSTRACT

The present invention relates to a brain cooling device for a craniotomy patient, and more specifically, to a brain cooling device for a craniotomy for cooling the brain so as to prevent damage to the brain after a brain surgery. To this end, the present invention provides the brain cooling device for a craniotomy patient comprising: a cooling portion which is buried in such a manner to come into contact with the brain or the dura mater; and one withdrawal tube, which is one end of the cooling portion that is exposed to the exterior, wherein the withdrawal tube is exposed to the exterior by penetrating through the scalp and the cranium. Otherwise, the present invention provides the brain cooling device for a craniotomy patient comprising: the cooling portion which is buried in such a manner to come into contact with the brain or the dura mater; and an introduction tube and a discharge tube, which are two end portions of the cooling portion that are exposed to the exterior, wherein the introduction tube and the discharge tube are exposed to the exterior by penetrating through the scalp and the cranium.

### CROSS-REFERENCE TO RELATED APPLICATION

This application claims priority to and the benefit of Korean Patent Application No. 10-2012-0015558, filed on Feb. 15, 2012, the disclosure of which is incorporated herein by reference in its entirety.

### BACKGROUND

1. Field of the Invention

The present invention relates to a brain cooling device for a craniotomy patient, and more particularly, to a brain cooling device for a craniotomy patient that cools the brain serving as a region for surgery to prevent edema and damage to the patient's brain immediately after the craniotomy.

2. Discussion of Related Art

In general, in the case of various types of brain damage such as a cerebral infarction, a cerebral hemorrhage, head trauma, etc., the implementation of therapeutic hypothermia to drop brain temperature may help to alleviate cerebral edema, increased intracranial pressure, and the like due to a disease.

Here, to drop the brain temperature, the brain temperature may drop due to the decreased temperature of the whole body, however, effectively lowering the brain temperature by directly lowering the brain temperature is impossible because the brain is encompassed by the cranium and the scalp.

The present invention, with an object to resolve the above-described problems, is directed to installing a brain cooling device through a region for surgery at the time of craniotomy, lowering the brain temperature during the period required after the surgery, and easily removing the brain cooling device inserted into the skull in a bed without further surgery once the therapeutic period has ended.

According to an aspect of the present invention, there is provided a brain cooling device for a craniotomy patient, comprising: a cooling portion which is buried in such a manner to come into contact with the brain or the dura mater; and one withdrawal tube, which is one end of the cooling portion exposed to the exterior, wherein the withdrawal tube is exposed to the exterior by penetrating through the scalp and the cranium.

According to another aspect of the present invention, there is provided a cooling portion which is buried in such a manner to come into contact with the brain or the dura mater; and an introduction tube and a discharge tube, which are two end portions of the cooling portion exposed to the exterior, wherein the introduction tube and the discharge tube are exposed to the exterior by penetrating through the scalp and the cranium.

According to another aspect of the present invention, the cooling portion may include soft tubes with a predetermined length.

According to another aspect of the present invention, the cooling portion may be configured to cover the upper part of the brain by forming multiple bent portions in a zigzag form.

According to another aspect of the present invention, a cooling material may be introduced or discharged through the withdrawal tube.

According to another aspect of the present invention, a cooling material may be introduced through the introduction tube and discharged through the withdrawal tube.

As described above, according to the brain cooling device for a craniotomy patient of the present invention, the cooling of brain can be effectively performed by installing a cooling portion that comes into direct contact with the brain or the dura mater, and using a cooling material which flows inside the cooling portion, and also the cooling portion can be easily removed in a bed.

### BRIEF DESCRIPTION OF THE DRAWINGS

FIG. 1 shows a brain cooling device for a craniotomy patient installed so that it can come into contact with the brain or the dura mater according to a first embodiment of the present invention;

FIG. 2 shows a schematic diagram of the internal structure of a cooling portion to be applied to the brain cooling device for a craniotomy patient according to the first embodiment of the present invention;

FIG. 3 shows a state of a fixed cranial bone flap after the completion of installing the brain cooling device for a craniotomy patient according to the first embodiment of the present invention;

FIG. 4 shows a state of a sutured scalp after the completion of installing the brain cooling device for a craniotomy patient according to the first embodiment of the present invention;

FIG. 5 shows a brain cooling device for a craniotomy patient installed so that it can come into contact with the brain or the dura mater according to a second embodiment of the present invention;

FIG. 6 shows a schematic diagram of the internal structure of a cooling portion to be applied to the brain cooling device for a craniotomy patient according to the second embodiment of the present invention;

FIG. 7 shows a state of a fixed cranial bone flap after the completion of installing the brain cooling device for a craniotomy patient according to the second embodiment of the present invention; and

FIG. 8 shows a state of a sutured scalp after the completion of installing the brain cooling device for a craniotomy patient according to the first embodiment of the present invention.

### DETAILED DESCRIPTION OF EXEMPLARY EMBODIMENTS

Exemplary embodiments of the present invention will be described in detail below with reference to the accompanying drawings.

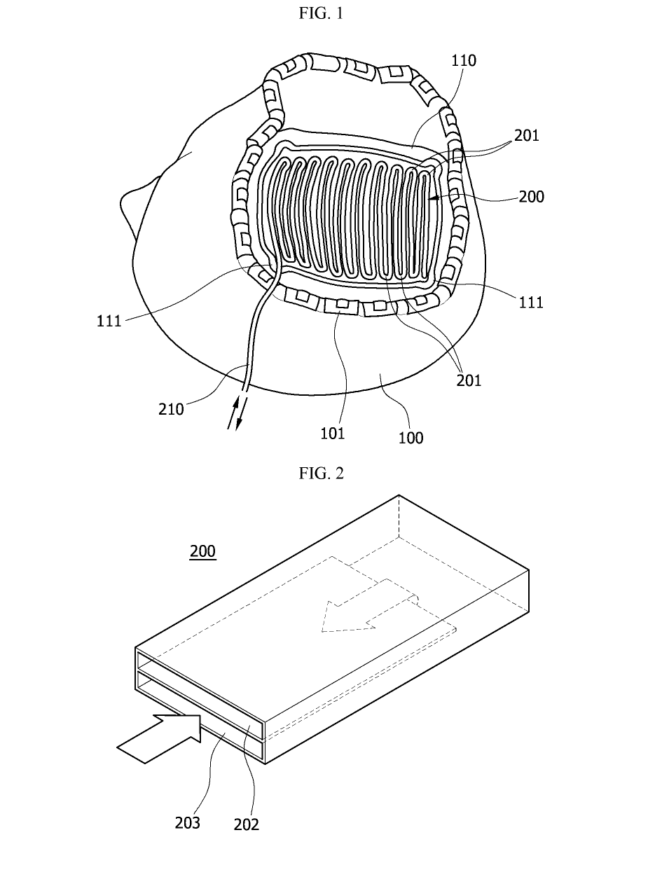

FIG. 1 shows a brain cooling device for a craniotomy patient installed so that it can come into contact with the brain or the dura mater according to a first embodiment of the present invention. As shown in FIG. 1, the brain cooling device for a craniotomy patient according to the first embodiment of the present invention includes a cooling portion 200, which is buried in such a manner to come into contact with the brain or the dura mater in a state in which a person's scalp 100 and cranium 110 are dissected and opened, and a withdrawal tube 210, which is exposed to the exterior by penetrating the scalp 100 and the cranium 110. In particular, the cooling portion 200 may be formed of a soft tube with a hollow center, i.e., in the form of a thin film formed of a material such as urethane or silicone.

Additionally, the cooling portion 200 is configured to form multiple bent portions in a zigzag form 201, and have a length sufficient to cover the upper portion of the brain. By installing it in this way, the area that comes in contact with the brain can be maximized, thereby more effectively performing the cooling activity.

Furthermore, the withdrawal tube 210 should be positioned through cranium perforations 111 formed during incision of the cranium 110, the penetration of the withdrawal tube 210 through the scalp 100 may be performed using a Trocar, and it is preferable that the withdrawal tube 210 be fixed around the scalp 100 using needles and threads for surgery.

Then, a cooling material is introduced and discharged through the withdrawal tube 210. Of course, the cooling material may be any material that enables a cooling activity by being subjected to heat exchange along with a physiological saline solution and nitrogen gas.

In addition, FIG. 2 shows a schematic diagram of the internal structure of a cooling portion to be applied to the brain cooling device for a craniotomy patient according to the first embodiment of the present invention. As shown in FIG. 2, if the cooling portion 200 is formed to be partitioned into an upper flow channel 202 and a lower flow channel 203, the cooling material may be introduced through one flow channel between the upper flow channel 202 and the lower flow channel 203, and at the same time, the cooling material may be discharged through the other flow channel, thereby minimizing the interference between introduction and discharge. As a result, the cooling efficiency can be improved.

FIG. 3 shows a state of a fixed cranial bone flap after the completion of installing the brain cooling device for a craniotomy patient according to the first embodiment of the present invention. As shown in FIG. 3, the withdrawal tube 210 may be exposed to the exterior by penetrating through the scalp using a Trocar having a predetermined distance from the sewing portion 102. In particular, the withdrawal tube 210 may be fixed using needles and threads for surgery near the perforated portion of the scalp. Accordingly, the brain can be maintained at a low temperature by introducing or discharging a cooling material through the withdrawal tube 210.

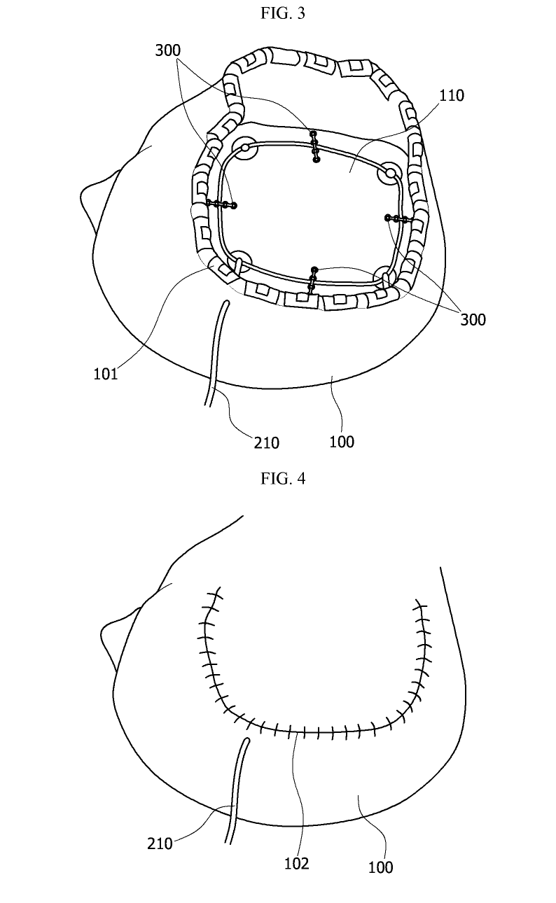

FIG. 4 shows a state of a sutured scalp after the completion of installing the brain cooling device for a craniotomy patient according to the first embodiment of the present invention. As shown in FIG. 4, when the scalp 100 is completely sealed by the sewing portion 102, the withdrawal tube 210 may remain exposed to the exterior. As such, the brain can be maintained at a low temperature by introducing or discharging a cooling material through the withdrawal tube 210. Later, the entire cooling portion 200 may be easily removed by cutting off the threads fixed to the withdrawal tube 210, and holding the withdrawal tube 210 with a hand and pulling it from the exterior of the scalp.

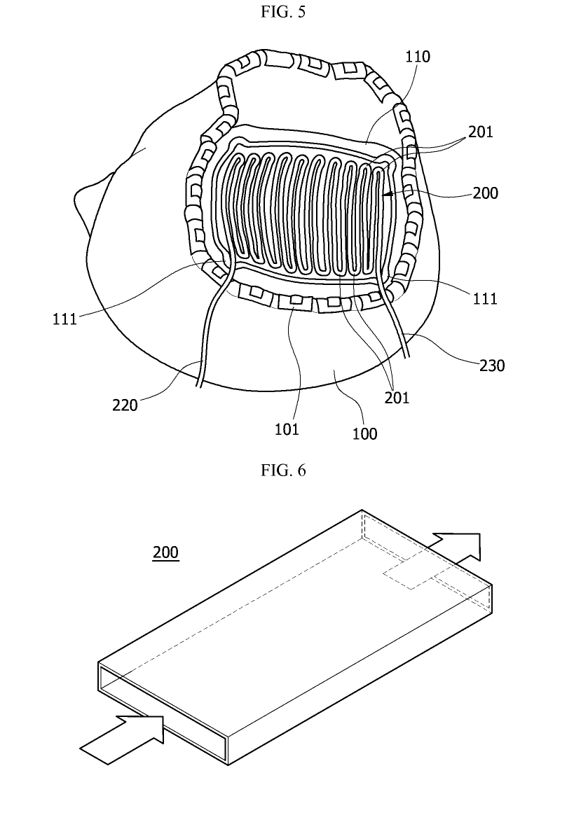

FIG. 5 shows a brain cooling device for a craniotomy patient installed so that it can come into contact with the brain or the dura mater according to a second embodiment of the present invention. As shown in FIG. 5, the brain cooling device for a craniotomy patient according to the second embodiment of the present invention has the same constitution as in the first embodiment described above in that the cooling portion 200 is buried in such a manner to come into contact with the brain or the dura mater in a state in which a person's scalp 100 and cranium 110 are dissected and opened. However, in the two end portions formed as an integral body extending from the cooling portion 200, the introduction tube 220 and the discharge tube 230 exposed to the exterior by penetrating through the scalp 100 and the cranium 110 are formed.

In particular, FIG. 6 shows a schematic diagram of the internal structure of a cooling portion to be applied to the brain cooling device for a craniotomy patient according to the second embodiment of the present invention. As shown in FIG. 6, although respective flow channels are formed inside the introduction tube 220 and the discharge tube 230, a cooling material is introduced through the introduction tube 220 and discharged through the discharge tube 230, and mutual interference between introduction and discharge at the time of introducing and discharging the cooling material can be prevented.

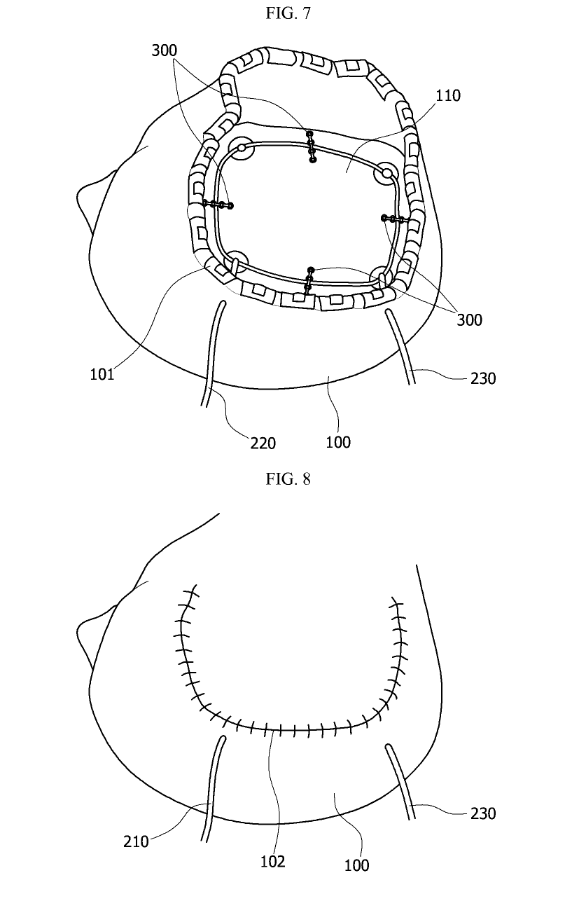

FIG. 7 shows a state of a sutured cranium after the completion of installing the brain cooling device for a craniotomy patient according to the second embodiment of the present invention. As shown in FIG. 7, if an already-open cranium 110 is closed and sealed tightly using an additionally provided binding member 300, the introduction tube 220 and the discharge tube 230 at the two ends that integrally extend from the cooling portion 200 are fixed to the perforations 111 of the cranium 110 and exposed to the exterior.

FIG. 8 shows a state of a sutured scalp after the completion of installing the brain cooling device for a craniotomy patient according to the first embodiment of the present invention. As shown in FIG. 8, the introduction tube 220 and the discharge tube 230 may be exposed to the exterior by penetrating through the scalp using a Trocar having a predetermined distance from the sewing portion 102. In particular, the introduction tube 220 and the discharge tube 230 are preferably fixed using needles and threads for surgery near the perforated portions of the scalp. By discharging a cooling material through the discharge tube 230 while simultaneously introducing the cooling material through the introduction tube 220, the cooling material may move at a faster flow rate, thereby further improving cooling performance. Later, the entire cooling portion 200 may be easily removed by cutting off any one of the threads fixed to introduction tube 220 and the discharge tube 230, and holding the other tube with a hand and pulling it from the exterior of the scalp.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

The brain cooling device of the present invention, when applied during the implementation of therapeutic hypothermia to alleviate cerebral edema, increased intracranial pressure, etc., in the case of various types of brain damage such as a cerebral infarction, a cerebral hemorrhage, head trauma, etc., helps to effectively lower brain temperature, and a cooling portion can be easily removed in a bed. The brain cooling device of the present invention may be used in the field of medical devices for improving the efficiency of the therapeutic hypothermia.

1. A brain cooling device for a craniotomy patient, comprising:
a cooling portion which is buried in such a manner to come into contact with the brain or the dura mater; and
one withdrawal tube, which is one end of the cooling portion that is exposed to the exterior, wherein the withdrawal tube is exposed to the exterior by penetrating through the scalp and the cranium.

2. A brain cooling device for a craniotomy patient, comprising:
a cooling portion which is buried in such a manner to come into contact with the brain or the dura mater; and
an introduction tube and a discharge tube, which are two end portions of the cooling portion that are exposed to the exterior, wherein the introduction tube and the discharge tube are exposed to the exterior by penetrating through the scalp and the cranium.

3. The brain cooling device according to claim 1, wherein the cooling portion comprises a soft tube with a predetermined length.

4. The brain cooling device according to claim 3, wherein the cooling portion is configured to cover the upper part of the brain by forming multiple bent portions in a zigzag form.

5. The brain cooling device according to claim 1, wherein a cooling material is introduced or discharged through the withdrawal tube.

6. The brain cooling device according to claim 2, wherein a cooling material is discharged through the withdrawal tube simultaneously as the cooling material is introduced through the introduction tube.

7. The brain cooling device according to claim 2, wherein the cooling portion comprises a soft tube with a predetermined length.

8. The brain cooling device according to claim 7, wherein the cooling portion is configured to cover the upper part of the brain by forming multiple bent portions in a zigzag form.
